SEQUENCE LISTING (2 sequences)

SEQ ID NO: 1 - protein, 228 aa, Homo sapiens
MDPNQNVKCKIVVVGDSQCGKTALLHVFAKDCFPENYVPTVFENYTASFEIDTQRIELSLWDTSGSPYYDNVRPLSYPDSDAVLICFDISRPETLDSVLKKWKGEIQEFCPNTKMLLVGCKSDLRTDVSTLVELSNHRQTPVSYDQGANMAKQIGAATYIECSALQSENSVRDIFHVATLACVNKTNKNVKRNKSQRATKRISHMPSRPELSAVATDLRKDKAKSCTV

SEQ ID NO: 2 - protein, 1354 aa, Homo sapiens
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

(19) United States
(12) Patent Application Publication
(10) Pub. No.: US 2014/0186277 A1
(43) Pub. Date: Jul. 3, 2014

(54) Methods and Compositions for Reducing Skin Damage

(71) Applicants: Shiseido Co., Ltd., Yokohama-shi (JP); The General Hospital Corporation, Boston, MA (US)

(72) Inventors: Sam W. Lee, Newton, MA (US); Lakshmi Raj, Boston, MA (US); Anna I. Mandinova, Newton, MA (US); Haruhi Iwaki, Kanagawa (JP); Yuji Katsuta, Kanagawa (JP); Jotaro Nakanishi, Kanagawa (JP)

(73) Assignees: Shiseido Co., Ltd., Yokohama-shi (JP); The General Hospital Corporation, Boston, MA (US)

(21) Appl. No.: 14/018,704

(22) Filed: Sep. 5, 2013

Related U.S. Application Data
(63) Continuation of application No. 12/742,804, filed on Dec. 13, 2010, now abandoned.
(60) Provisional application No. 61/003,351, filed on Nov. 15, 2007.

(51) Int. Cl. A61K 31/353

(52) U.S. Cl. CPC A61K 31/353
USPC 424/59; 514/456; 549/403

(57) ABSTRACT

The RhoE GTPase pathway has been identified as a target for screening and treatment methods for the prevention and/or reduction of short- and long-term UVB-induced skin damage, e.g., the prevention and/or reduction of UVB-induced wrinkles. The invention thus features screening and treatment methods for prevention or reduction of UVB-induced skin damage, and related compositions, e.g., cosmetic compositions.

### CROSS-REFERENCE TO RELATED APPLICATIONS

This application claims priority to U.S. application Ser. No. 61/003,351, filed on Nov. 15, 2007, which is incorporated herein by reference in its entirety.

### BACKGROUND

Members of the Rho family of Ras-related GTPases, such as RhoE, regulate the organization of the actin cytoskeleton in response to extracellular growth factors.

### SUMMARY

The invention is based, in part, on the discovery that the RhoE pathway is important for the maintenance and/or appearance of skin. In one embodiment, the inventors have found that the RhoE pathway is important in the reduction, treatment, and/or prevention of skin damage, e.g., ultraviolet B (UVB)-induced skin damage and wrinkles. Therefore, the inventors have identified the RhoE pathway as a target for screening and therapeutic methods to improve condition and/or appearance of skin, e.g., by the prevention and/or reduction of acute and/or chronic photodamage, e.g., UVB-induced skin damage (e.g., the prevention and/or reduction of wrinkles). The invention thus features screening and treatment methods improve condition and/or appearance of skin, e.g., by reduction, treatment, and/or prevention of UVB-induced skin damage, e.g., wrinkles, and related compositions, e.g., cosmetic compositions.

Accordingly, in one aspect, the invention features a method of screening for an agent that prevents and/or reduces UVB-induced skin damage, e.g., wrinkles. The method includes identifying any agent that increases RhoE pathway activity, e.g., increases RhoE activity, induces RhoE expression, decreases Rho kinase I (ROCK I) activity, or reduces ROCK I expression.

The method can also include associating increased RhoE pathway activity (e.g., increased RhoE activity, increased RhoE expression, decreased ROCK I activity, or decreased ROCK I expression) with the agent's ability to prevent and/or reduce wrinkles, e.g., identifying the identified agent as a wrinkle protection and/or reduction agent (e.g., providing print material or a computer readable medium, e.g., informational, marketing, or instructional print material or computer readable medium, related to the identified agent or its use). Associating means identifying a test agent that increases RhoE pathway activity as an agent capable of preventing, reducing and/or treating wrinkles. The associating step can include, e.g., generating or providing a record, e.g., a print or computer readable record, such as a laboratory record or dataset or an email, identifying a test agent that increases RhoE pathway activity as an agent capable of preventing, reducing and/or treating wrinkles. The record can include other information, such as a specific test agent identifier, a date, an operator of the method, or information about the source, structure, method of purification, or biological activity of the test agent. The record or information derived from the record can be used, e.g., to identify the test agent as a compound or candidate agent (e.g., a lead compound) for pharmaceutical or therapeutic use. The identified agent can be identified as an agent or a potential agent for treatment and/or reduction of wrinkles. Agents, e.g., compounds, identified by this method can be used, e.g., in the treatment (or development of treatments, e.g., cosmetic treatments) for wrinkles.

In one embodiment, the method includes evaluating, e.g., measuring, the effect of the agent on skin, e.g., evaluating a parameter correlated with wrinkles, e.g., the presence, extent, or type of wrinkles; and selecting an agent from the screen, e.g., an agent that prevents and/or reduces damage to the skin, e.g., prevents and/or reduces wrinkles in the skin. Preferably, evaluating the effect of the agent on skin includes administering the agent, e.g., topically, to a tissue or subject and comparing a parameter correlated with wrinkles, e.g., the presence, extent, or type of wrinkles in the tissue or subject, optionally with a reference value, e.g., a control or baseline value, e.g., a value for the same parameter in a tissue or subject that has been treated differently, e.g., has not been administered the agent or has been administered a placebo. The effect of the agent on skin can be evaluated in the absence or presence of a source of skin damage, e.g., an agent or treatment that induces wrinkle formation, e.g., UVB radiation. In some embodiments, the evaluation includes entering a value for the evaluation, e.g., a value for the presence, extent, or type of wrinkles into a database or other record.

In one embodiment, an agent is evaluated for the ability to prevent and/or reduce UVB-induced wrinkles.

In another embodiment, the subject is an experimental animal e.g., a wild-type or transgenic experimental animal, e.g., a rodent, e.g., a rat, mouse or guinea pig. The subject can also be a human. In a further embodiment, the evaluating step comprises administering the agent to the skin of the subject, e.g., topically.

In one embodiment, an agent that increases RhoE pathway activity (e.g., increases RhoE activity, induces RhoE expression, decreases ROCK I activity, or reduces ROCK I expression) is identified.

In another embodiment, the identifying step includes: (a) providing a cell, tissue or non-human animal harboring an exogenous nucleic acid that includes a regulatory region (e.g., a promoter or enhancer) of a component of the RhoE pathway (e.g., RhoE or ROCK I) operably linked to a nucleotide sequence encoding a reporter polypeptide (e.g., a light based, e.g., luminescent (e.g., luciferase) colorimetric, or fluorescently detectable (e.g., a fluorescent reporter polypeptide, e.g., GFP, EGFP, BFP, RFP) label; (b) evaluating the ability of a test agent to increase the activity of the reporter polypeptide in the cell, tissue or non-human animal; and (c) selecting a test agent that modulates the activity of the reporter polypeptide (e.g., relative to a reference control) as an agent that modulates a component of the RhoE pathway. In one embodiment, the cell or tissue is a skin cell or tissue, e.g., a keratinocyte cell or tissue, e.g., a skin explant or artificial skin tissue. In another embodiment, the non-human animal is a transgenic animal, e.g., a transgenic rodent, e.g., a mouse, rat, or guinea pig, harboring the nucleic acid.

In one embodiment, the method includes two evaluating steps, e.g., the method includes a first step of evaluating the test agent in a first system, e.g., a cell or tissue system, and a second step of evaluating the test agent in a second system, e.g., a second cell or tissue system or in an non-human animal. In other embodiments, the method includes two evaluating steps in the same type of system, e.g., the agent is re-evaluated in a non-human animal after a first evaluation in the same or a different non-human animal. The two evaluations can be separated by any length of time, e.g., days, weeks, months or years.

In another embodiment, the effect of the agent on UVB-induced wrinkles is evaluated. For example, the agent is evaluated before, during, and/or after UVB exposure.

An agent that modulates the expression, activity, and/or levels of a component of the RhoE pathway, e.g., RhoE, can be a crude or semi-purified extract, e.g., an organic, e.g., animal or botanic extract, or an isolated compound, e.g., a small molecule, protein, lipid, or nucleic acid. Typical agents are naturally occurring substances or extracts, e.g., plant or fungal extract. For example, the agent can be any of: (a) a polypeptide component of the RhoE pathway, e.g., a RhoE polypeptide or a functional fragment or mimetic thereof; (b) a peptide or protein agonist or antagonist of a component of the RhoE pathway that increases an activity of the RhoE pathway; (c) a small molecule or chemical compound (e.g., an organic compound, e.g., a naturally occurring or synthetic organic compound) that increases expression of a component of the RhoE pathway, e.g., RhoE, e.g., by binding to the promoter region of its gene; (d) a small molecule that decreases expression of a component of the RhoE pathway, e.g., ROCK I, e.g., by binding to the promoter region of its gene; (f) a nucleotide sequence encoding a RhoE pathway polypeptide or functional fragment, analog, activated allele, or activator thereof; or (g) a nucleotide sequence (e.g., an antisense, siRNA, dsRNA, or hairpin nucleic acid) that decreases expression of a RhoE pathway polypeptide (e.g., ROCK I). The nucleotide sequence can be a genomic sequence or a cDNA sequence. The nucleotide sequence, e.g., a viral vector (e.g., an adenovirus vector, an adeno-associated virus vector, a retrovirus vector, or a lentivirus vector), can include: a RhoE pathway component coding region; a promoter sequence, e.g., a promoter sequence from a RhoE pathway component gene or from another gene; an enhancer sequence; untranslated regulatory sequences, e.g., a 5′ untranslated region (UTR), e.g., a 5′ UTR from a RhoE gene or from another gene, a 3′ UTR, e.g., a 3′ UTR from a RhoE gene or from another gene; a polyadenylation site; and/or an insulator sequence. In another embodiment, the level of a component of the RhoE pathway, e.g., RhoE, is increased by increasing the level of expression of an endogenous component of the RhoE pathway, e.g., by increasing, transcription of the RhoE gene or increasing RhoE mRNA stability. In one embodiment, transcription of the RhoE gene is increased by: altering the regulatory sequence of the endogenous factor RhoE gene, e.g., in a somatic cell, e.g., by the addition of a positive regulatory element (such as an enhancer or a DNA-binding site for a transcriptional activator); the deletion of a negative regulatory element (such as a DNA-binding site for a transcriptional repressor) and/or replacement of the endogenous regulatory sequence, or elements therein, with that of another gene, thereby allowing the coding region of the RhoE gene to be transcribed more efficiently. In another embodiment, the agent is in a crude or partially purified botanical extract.

One or more agents can be used in combination for the methods described herein.

In one aspect, the invention features methods of reducing, treating, and/or preventing skin damage, e.g., UVB-induced skin damage and/or wrinkles, by administering to the subject an agent that increases RhoE pathway activity (e.g., increases RhoE activity, induces RhoE expression, decreases ROCK I activity, or reduces ROCK I expression) in an amount sufficient to reduce, treat, and/or prevent skin damage. In some embodiments, the agent modulates a component of the RhoE pathway, e.g., RhoE. In some embodiments, the methods further include identifying a subject in need of reduction, treatment, and/or prevention of skin damage. In some embodiments, the agent is administered topically. In some embodiments, the subject has been or will be exposed to UVB radiation (e.g., a skin-damaging amount of UVB radiation). In some embodiments, the agent is selected from those presented in Table 1. Other agonists of the RhoE pathway can also be used in the methods.

In yet another aspect, the invention features methods of reducing one or more signs of skin damage, e.g., one or more wrinkling (e.g., number or morphology of wrinkles), redness, inflammation, desquamation, and pigmentation, in a subject by administering to the subject an agent that increases RhoE pathway activity (e.g., increases RhoE activity, induces RhoE expression, decreases ROCK I activity, or reduces ROCK I expression) in an amount sufficient to reduce wrinkles, e.g., wrinkles caused by exposure to UVB radiation. In some embodiments, the agent modulates a component of the RhoE pathway, e.g., RhoE. In some embodiments, the agent is selected from those presented in Table 1. In some embodiments, the agent is administered topically. The agent can be in a composition, e.g., cosmetic composition. The composition can be sterile and/or it can further include a cosmetic agent.

In another aspect, the invention features methods of protecting against skin damage, e.g., UVB-induced skin damage in a subject by supplying to a subject a composition that includes an agent that increases RhoE pathway activity (e.g., increases RhoE activity, induces RhoE expression, decreases ROCK I activity, or reduces ROCK I expression) in an amount sufficient to protect against skin damage. In some embodiments, the composition modulates a component of the RhoE pathway. In some embodiments, the methods further include supplying to the subject instructions for using the compositions to protect against skin damage, e.g., UVB-induced skin damage and/or wrinkles. In some embodiments, the instructions include directions to apply the composition to the skin prior to, during, and/or after sun exposure. The composition can include a RhoE pathway agonist, e.g., an agent selected from those presented in Table 1. The composition can include a cosmetic agent.

In another aspect, the invention features methods of maintaining skin homeostatis in a subject by administering to the subject an agent that increases RhoE pathway activity (e.g., increases RhoE activity, induces RhoE expression, decreases ROCK I activity, or reduces ROCK I expression), thereby maintaining skin homeostatis. In some embodiments, the agent is administered topically. In some embodiments, the subject has been or will be exposed to UVB radiation (e.g., a skin-damaging amount of UVB radiation). In some embodiments, the agent is selected from those presented in Table 1.

In another aspect, the invention features methods of normalizing epidermal function in a subject by administering to the subject an agent that activates RhoE pathway activity (e.g., increases RhoE activity, induces RhoE expression, decreases ROCK I activity, or reduces ROCK I expression), thereby normalizing epidermal function. In some embodiments, the agent is administered topically. In some embodiments, the subject has been or will be exposed to UVB radiation (e.g., a skin-damaging amount of UVB radiation). In some embodiments, the agent is selected from those presented in Table 1.

In another aspect, the invention features methods of preventing, reducing, and/or treating skin aging in a subject by administering to the subject an agent that increases RhoE pathway activity (e.g., increases RhoE activity, induces RhoE expression, decreases ROCK I activity, or reduces ROCK I expression), thereby preventing, reducing, or treating skin aging. In some embodiments, the agent is administered topically. In some embodiments, the subject has been or will be exposed to UVB radiation (e.g., a skin-damaging amount of UVB radiation). In some embodiments, the agent is selected from those presented in Table 1.

In another aspect, the invention features methods of improving barrier function of the skin of a subject by administering to the subject an agent that increases RhoE pathway activity (e.g., increases RhoE activity, induces RhoE expression, decreases ROCK I activity, or reduces ROCK I expression), thereby improving barrier function of the skin. In some embodiments, the agent is administered topically. In some embodiments, the subject has been or will be exposed to UVB radiation (e.g., a skin-damaging amount of UVB radiation). In some embodiments, the agent is selected from those presented in Table 1.

In another aspect, the invention features methods of preventing, reducing, and/or treating dry skin by administering to the subject an agent that increases RhoE pathway activity (e.g., increases RhoE activity, induces RhoE expression, decreases ROCK I activity, or reduces ROCK I expression), thereby preventing, reducing, or treating dry skin. In some embodiments, the agent is administered topically. In some embodiments, the subject has been or will be exposed to UVB radiation (e.g., a skin-damaging amount of UVB radiation). In some embodiments, the agent is selected from those presented in Table 1.

In another aspect, the invention features methods of preventing, reducing, and/or treating wrinkle formation by administering to the subject an agent that increases RhoE pathway activity (e.g., increases RhoE activity, induces RhoE expression, decreases ROCK I activity, or reduces ROCK I expression), thereby preventing, reducing, or treating wrinkle formation. In some embodiments, the agent is administered topically. In some embodiments, the subject has been or will be exposed to UVB radiation (e.g., a skin-damaging amount of UVB radiation). In some embodiments, the agent is selected from those presented in Table 1.

The term “RhoE pathway” refers to the biological components that mediate the effects of RhoE on apoptosis and differentiation (see FIG. 9). The pathway includes, e.g., RhoE and ROCK I. The term “RhoE pathway agonist” refers to an agent that increases activity of the RhoE pathway, e.g., an agent that potentiates, induces, or otherwise enhances one or more biological activities of a RhoE polypeptide, e.g., a biological activity as described herein.

In one embodiment, the RhoE pathway agonist is a nucleic acid that encodes a RhoE polypeptide or a positively acting cytoplasmic pathway component. In another embodiment, the RhoE pathway agonist is a nucleic acid that inhibits expression of ROCK I (e.g., an antisense or RNAi nucleic acid).

The subject can be mammalian, and typically is human (e.g., a female or a male, and an adult or a juvenile human subject).

The method can further include evaluating one or more signs of skin damage in the subject, e.g., before, during, or after the administering. Examples of such signs are described herein. The method can further include evaluating a RhoE associated parameter in the subject, e.g., a parameter associated with level of RhoE polypeptide. RhoE receptor, or RhoE pathway activity. The term “parameter” refers to information, including qualitative and quantitative descriptors, e.g., values, levels, measurements, and so forth. A “RhoE associated parameter” refers to a parameter that describes a RhoE pathway component, e.g., the presence, absence, level, expression, stability, subcellular localization, or activity of such a component, e.g., a RhoE polypeptide or other cytoplasmic component. The parameter may also describe an mRNA that encodes a RhoE pathway component.

In another aspect, the invention features compositions, e.g., cosmetic compositions, that include an agent that increases RhoE pathway activity (e.g., increases RhoE activity, induces RhoE expression, decreases ROCK I activity, or reduces ROCK I expression). In some embodiments the agent modulates a component of the RhoE pathway, e.g., RhoE. The cosmetic compositions can further include a second ingredient, e.g., a cosmetic ingredient (e.g., a fragrance, moisturizer, or sunscreen). In some embodiments, the agent that modulates a component of the RhoE pathway is selected from those presented in Table 1. These compositions can be used in methods for the treatment and/or prevention of skin damage, e.g., UVB-induced skin damage and/or wrinkles.

In another aspect, the invention features compositions, e.g., compositions for topical application, that include an agent that increases RhoE pathway activity (e.g., increases RhoE activity, induces RhoE expression, decreases ROCK I activity, or reduces ROCK I expression), in an amount sufficient to reduce skin damage, e.g., UVB-induced skin damage and/or wrinkles. In some embodiments, the agent modulates a component of the RhoE pathway. The agent can be, e.g., selected from those presented in Table 1. The composition can further include a cosmetic ingredient, e.g., a fragrance or sunscreen.

In another aspect, the invention features the use of an effective amount of an agent that increases RhoE pathway activity (e.g., increases RhoE activity, induces RhoE expression, decreases ROCK I activity, or reduces ROCK I expression) in the preparation of a medicament or cosmetic for preventing, reducing, and/or treating skin damage, e.g., UVB-induced skin damage and/or wrinkles or the use of an effective amount of an agent that increases RhoE pathway activity (e.g., increases RhoE activity, induces RhoE expression, decreases ROCK I activity, or reduces ROCK I expression) for preventing, reducing, and/or treating skin damage, e.g., UVB-induced skin damage and/or wrinkles. In some embodiments, the agent modulates a component of the RhoE pathway. The agent can be, e.g., selected from those presented in Table 1. The medicament or cosmetic can further include a cosmetic ingredient, e.g., a fragrance or sunscreen.

In yet another aspect, the invention features kits for reducing, treating, and/or preventing skin damage, e.g., UVB-induced skin damage and/or wrinkles, in a subject that include a composition that includes an agent that increases RhoE pathway activity, levels, or expression, e.g., an agent modulates a component of the RhoE pathway, and instructions for using the composition to prevent skin damage. The composition can further include a cosmetic ingredient, e.g., a fragrance or sunscreen. The instructions can include directions to apply the composition to the skin prior to, during, or after sun exposure.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the claims. All references cited herein are incorporated by reference.

### DESCRIPTION OF DRAWINGS

FIG. 1A is a schematic diagram depicting the RhoE luciferase reporter.

FIG. 1B is a flowchart of the method of screening for inducers of RhoE expression.

FIG. 2 is a graph depicting the distribution of Z values for two independent replicates of each compound.

FIG. 3 is a Western blot depicting RhoE and p53 expression following treatment with the indicated amounts of kaempferol.

FIG. 4 is a bar graph depicting the rate of cell death of untreated and kaempferol-treated cells following UVB exposure.

FIG. 5 is a set of micrographs depicting apoptosis in mouse skin following UVB exposure and treatment with 0.5 mM kaempferol. TUNEL staining is indicated in red and DAPI staining (for nuclei) is indicated in blue.

FIGS. 6A-6C are micrographs of DNA damage in mouse skin following UVB exposure and treatment with 0.5 mM kaempferol (6B) or DMSO control (6A). 6C shows a control without UV exposure. TDM-2 staining is indicated in green and DAPI staining is indicated in blue.

FIGS. 7A-7C are micrographs of mouse skin following UVB exposure and treatment with 0.5 mM kaempferol (7B) or DMSO control (7A). 7C shows a control without UV exposure. Anti-phospho-H2AX staining is indicated in red and DAPI staining is indicated in blue.

FIG. 8 is a set of micrographs depicting RhoE expression in mouse epidermis following UVB exposure and treatment with 0.5 mM kaempferol. DAPI staining is indicated in blue and RhoE staining is indicated in red.

FIG. 9 is a model of the role of RhoE and ROCK I in survival, apoptosis, and differentiation of keratinocytes.

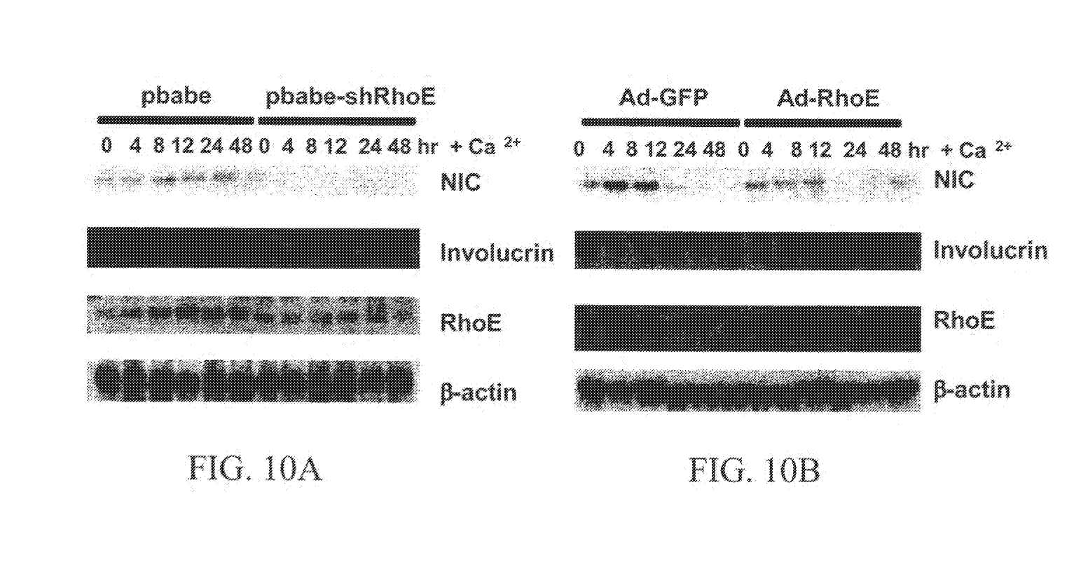

FIGS. 10A-10B are Western blots depicting NIC, Involucrin, RhoE, and β-actin control following: 10A, inhibition of RhoE expression (pbabe-shRhoE) compared to vector control (pbabe); or 10B, overexpression of RhoE (Ad-RhoE) compared to control (Ad-GFP).

FIGS. 11A-11B are micrographs depicting expression of keratin 1 in epidermis of wild-type (11A) and RhoE transgenic (11B) mice. Keratin 1 is indicated in red and DAPI is indicated in blue.

FIGS. 12 and 13 are sets of micrographs depicting RhoE expression in human skin following UVB exposure.

FIG. 14 is a bar graph depicting relative RhoE mRNA levels in human keratinocytes as measured by quantitative PCR following exposure to the medicated levels of UVB.

### DETAILED DESCRIPTION

The inventors have identified the RhoE pathway as a target for screening and therapeutic methods as well as compositions, e.g., cosmetic compositions, for treatment, prevention, and/or reduction of skin damage, e.g., UVB-induced skin damage, e.g., wrinkles. This invention features compositions having a RhoE inducer as an active ingredient.

### Skin Damage

Damaged skin is typically characterized by one or more of inflammation, epidermal hyperplasia, dermal elastosis and matrix protein degradation, and the presence of perivenular lymphohistiocytic dermal infiltrates. Results described herein reveal that RhoE protects against skin damage, e.g., skin damage caused by UVB radiation.

An effective amount of a composition is defined as the amount of the composition which, upon administration to a subject, prevents or reduces one or more signs of skin damage, e.g., inflammation, epidermal hyperplasia, dermal elastosis and matrix protein degradation, perivenular lymphohistiocytic dermal infiltrates, and the formation of wrinkles (e.g., fine wrinkles), in the subject. The effective amount to be administered to a subject is typically based on a variety of factors including age, sex, surface area, weight, and conditions of the skin. Body surface area may be approximately determined from height and weight of the patient. See, e.g., Scientific Tables, Geigy Pharmaceuticals, Ardley, N.Y., 1970, 537. Effective doses will vary, as recognized by those skilled in the art, dependent on route for administration, excipient usage, and the possibility of co-usage with other treatments such as usage of other wrinkle reducing compounds. An experimental animal can be used in a method of determining an effective amount of a composition.

As used herein, “preventing or treating skin damage” means the application or administration of a therapeutic agent to a subject who has skin damage, e.g., a wrinkle, or has a predisposition toward skin damage, or has been exposed to an agent likely to cause skin damage, e.g., UV radiation, e.g., UVB radiation, with the purpose to reduce, improve, alleviate, alter, remedy, ameliorate, or affect the appearance of skin damage. The therapeutic agent can be administered to the subject by the subject himself or herself, or by another person, e.g., a health care provider or a provider of cosmetics. In preferred embodiments of the methods described herein, skin damage is reduced in the subject by at least 5%, typically at least 10%, e.g., at least 20%, 25% or more.

The methods and compositions can be used prophylactically or they can be used to reduce, treat, and/or prevent further skin damage, e.g., wrinkles, or reduce the appearance of skin damage in a subject. The composition can also be used for the manufacture of a medicament or cosmetic for preventing, reducing, and/or treating skin damage, e.g., wrinkles.

### Wrinkles

Wrinkles are generally a result of the natural aging process of the skin and of exposure to the sun's ultraviolet rays. A wrinkle is a configuration change in the surface of the skin, without specific structural alterations at the histological level. Generally, wrinkles are classified as described in Ligman et al. (1985) Br. J. Derm. 113:37-42, incorporated herein by reference. Kligman classifies wrinkles into three classes: linear wrinkles, glyphic wrinkles, and crinkles. Linear wrinkles are straight, found generally in the facial skin, and are caused by natural aging or exposure to ultraviolet light. Glyphic wrinkles are shaped as apparent triangles or rectangles of wrinkles, are found on the face, hands, and neck exposed to sunlight, and are aggravated by exposure to ultraviolet light or dermatoheliosis. Crinkles, are thin, crinkled wrinkles on flabby skin, found anywhere on the skin, but typically on the backs of hands and around the eyelids.

Linear wrinkles can be further subclassified into (a) regular wrinkles and (b) fine wrinkles. Regular wrinkles are long, deep, clear, and are also referred to as crow's feet. Fine wrinkles are thin and shallow. Regular wrinkles have a width of at least about 155 microns (0-32 Hz), typically about 160 to 250 microns. Fine wrinkles have a width of less than about 154 microns, typically about 40 to 154 microns (32-126 Hz), as calculated, e.g., in a power spectrum obtained through transforming three dimensional shape data into data in a frequency domain by two-dimensional Fourier transformation (using, e.g., the Shiseido Wrinkle Analyzer 3D Pro system, essentially as described in Takasu et al. (1996) J. Soc. Cosmet. Chem. Japan 29:394-405; and Japanese Published Patent Application No. 07-113623, published May 2, 1995).

### Methods of Screening

The RhoE pathway, including RhoE and ROCK I is described in Ongusaha et al. (2006) Curr. Biol. 16:2466-72 and Boswell et al. (2007) J. Biol. Chem. 282:4850-58, both of which are incorporated herein by reference. The components of the pathway have been cloned from multiple species, and their protein and gene sequences are readily available to one of ordinary skill in the art. RhoE sequences are available from e.g., human, chimpanzee, rhesus monkey, mouse, rat, cattle, and horse. ROCK-I sequences are available from, e.g., human, chimpanzee, rhesus monkey, mouse, rat, dog, rabbit, cattle, and horse.

An exemplary human RhoE polypeptide is provided as SEQ ID NO:1.

(SEQ ID NO: 1)
MDPNQNVKCKIVVVGDSQCGKTALLHVFAKDCFPENYVPTVFENYTASF
EIDTQRIELSLWDTSGSPYYDNVRPLSYPDSDAVLICFDISRPETLDSV
LKKWKGEIQEFCPNTKMLLVGCKSDLRTDVSTLVELSNHRQTPVSYDQG
ANMAKQIGAATYIECSALQSENSVRDIFHVATLACVNKTNKNVKRNKSQ
RATKRISHMPSRPELSAVATDLRKDKAKSCTVM
An exemplary human ROCK1 polypeptide is provided
as SEQ ID NO: 2.
(SEQ ID NO: 2)
MSTGDSFETRFEKMDNLLRDPKSEVNSDCLLDGLDALVYDLDFPALRKN
KNIDNFLSRYKDTINKIRDLRMKAEDYEVVKVIGRGAFGEVQLVRHKST
RKVYAMKLLSKFEMIKRSDSAFFWEERDIMAFANSPWVVQLFYAFQDDR
YLYMVMEYMPGGDLVNLMSNYDVPEKWARFYTAEVVLALDAIHSMGFIH
RDVKPDNMLLDKSGHLKLADFGTCMKMNKEGMVRCDTAVGTPDYISPEV
LKSQGGDGYYGRECDWWSVGVFLYEMLVGDTPFYADSLVGTYSKIMNHK
NSLTFPDDNDISKEAKNLICAFLTDREVRLGRNGVEEIKRHLFFKNDQW
AWETLRDTVAPVVPDLSSDIDTSNFDDLEEDKGEEETFPIPKAFVGNQL
PFGVFTYYSNRRYLSSANPNDNRTSSNADKSLQESLQKTIYKLEEQLHN
EMQLKDEMEQKCRTSNIKLDKIMKELDEEGNQRRNLESTVSQIEKEKML
LQHRINEYQRKAEQENEKRRNVENEVSTLKDQLEDLKKVSQNSQLANEK
LSQLQKQLEEANDLLRTESDTAVRLRKSHTEMSKSISQLESLNRELQER
NRILENSKSQTDKDYYQLQAILEAERRDRGHDSEMIGDLQARITSLQEE
VKHLKHNLEKVEGERKEAQDMLNHSEKEKNNLEIDLNYKLKSLQQRLEQ
EVNEHKVTKARLTDKHQSIEEAKSVAMCEMEKKLKEEREAREKAENRVV
QIEKQCSMLDVDLKQSQQKLEHLTGNKERMEDEVKNLTLQLEQESNKRL
LLQNELKTQAFEADNLKGLEKQMKQEINTLLEAKRLLEFELAQLTKQYR
GNEGQMRELQDQLEAEQYFSTLYKTQVKELKEEIEEKNRENLKKIQELQ
NEKETLATQLDLAETKAESEQLARGLLEEQYFELTQESKKAASRNRQEI
TDKDHTVSRLEEANSMLTKDIEILRRENEELTEKMKKAEEEYILEKEEE
ISNLKAAFEKNINTERTLKTQAVNKLAEIMNRKDFKIDRKKANTQDLRK
KEKENRKLQLELNQEREKFNQMVVKHQKELNDMQAQLVEECAHRNELQM
QLASKESDIEQLRAKLLDLSDSTSVASFPSADETDGNLPESRIEGWLSV
PNRGNIKRYGWKKQYVVVSSKKILFYNDEQDKEQSNPSMVLDIDKLFHV
RPVTQGDVYRAETEEIPKIFQILYANEGECRKDVEMEPVQQAEKTNFQN
HKGHEFIPTLYHFPANCDACAKPLWHVFKPPPALECRRCHVKCHRDHLD
KKEDLICPCKVSYDVTSARDMLLLACSQDEQKKWVTHLVKKIPKNPPSG
FVRASPRTLSTRSTANQSFRKVVKNTSGKTS

Naturally occurring and synthetic variants of these or other RhoE and ROCK-I sequences can be used in the methods described herein.

Numerous methods exist for evaluating whether an agent alters the expression, levels, or activity of a particular mRNA or protein. In one embodiment, the ability of a test agent to modulate (e.g., increase or decrease) (e.g., permanently or temporarily) expression from a RhoE pathway promoter (e.g., the RhoE promoter) is evaluated by, e.g., reporter (e.g., luciferase, LacZ, or GFP) transcription assay. For example, a cell or transgenic animal, the genome of which comprises a reporter gene operably linked to a RhoE pathway promoter, can be contacted with a test agent, and the ability of the test agent to increase or decrease reporter activity is indicative of the ability of the agent to modulate RhoE pathway activity. In another embodiment, the ability of a test agent to modulate RhoE expression, levels, or activity is evaluated in a transgenic animal. The effect of a test agent on RhoE expression, levels, or activity, may be evaluated on a cell, cell lysate, or subject, typically, a non-human experimental mammal, e.g., a rodent (e.g., a rat, mouse, rabbit), or explant (e.g., skin) thereof. Numerous methods of assessing mRNA expression are well know in the art, e.g., Northern analysis, ribonuclease protection assay, reverse transcription-polymerase chain reaction (RT-PCR) or RNA in situ hybridization (see, e.g., Sambrook et al. Molecular Cloning: A Laboratory Manual (3^{rd} ed. 2001)). Protein levels may be monitored by, e.g., Western analysis, immunoassay, or in situ hybridization. General methods of small-molecule screening are discussed in, e.g., Schrieber (2003) Chem. & Eng. News 81:51-61; and Flaumenhaft and Sim (2003) Chem. Biol. 10:481-6.

Nucleic acids described herein can be contained within a vector, e.g., as a virus that includes a nucleic acid that expresses the nucleic acid. Exemplary viral vectors include adenoviruses (reviewed in Altaras et al., 2005, Adv. Biochem. Eng. Biotechnol., 99:193-260), adeno-associated viruses (reviewed in Park et al., 2008, Front. Biosci., 13:2653-59; see also Williams, 2007 Mol. Ther., 15:2053-54), parvoviruses, lentiviruses, retroviruses (reviewed in tai et al., 2008, Front. Biosci., 13:3083-95), and the herpes simplex virus. Method of delivery of nucleic acids are reviewed in Patil et al., 2005, AAPS J., 7:E61-77, which is incorporated herein by reference in its entirety.

### Agents

Agents to be tested in the screening methods described herein include crude or purified extracts of organic sources, e.g., animal or botanical extracts, as well as partially or fully purified or synthetic agents, e.g., small molecules, polypeptides, lipids and/or nucleic acids, and libraries of these. Agents that are identified as activators of RhoE pathway activity can be tested and/or used in the skin damage-related methods and compositions described herein. Several agents that activate RhoE pathway activity (e.g., induce RhoE expression are identified herein and presented in Table 1. These and structurally or functionally similar agents can be tested and/or used in the treatment of, e.g., skin damage and wrinkles.

### Administration

The compositions for the prevention or reduction of wrinkles or other skin conditions, or for the treatment of other disorders described herein, may be administered via the parenteral route, including topically, subcutaneously, intraperitoneally, intramuscularly, intranasally, and intravenously. Topical administration is typically used. Repeated administration of the composition, e.g., repeated topical administration, can be used. More than one route of administration can be used simultaneously, e.g., topical administration in association with oral administration. Examples of parenteral dosage forms include aqueous solutions of the active agent in a isotonic saline, 5% glucose, or other well-known pharmaceutically acceptable excipient. Solubilizing agents, such as cyclodextrins or other solubilizing agents, can be utilized as pharmaceutical excipients for delivery of the wrinkle reducing composition.

A composition described herein can also be formulated into dosage forms for other routes of administration utilizing conventional methods. A pharmaceutical composition can be formulated, for example, in dosage forms for oral administration in a capsule, a tablet (each including timed release and sustained release formulations), or a gel seal. Capsules may comprise any standard pharmaceutically acceptable material such as gelatin or cellulose derivatives. Tablets may be formulated in accordance with the conventional procedure by compressing mixtures of an agent and a solid carrier, and a lubricant. Examples of solid carriers include starch and sugar bentonite. A pharmaceutical composition can also be administered in a form of a hard shell tablet or capsule containing, for example, lactose or mannitol as a binder and a conventional filler and a tableting agent.

Topical administration of the compounds, e.g., wrinkle-reducing compounds, described herein presents a preferred route of administration amongst the many different routes described above. For topical application, the composition can include a medium compatible with skin. Such topical pharmaceutical compositions can exist in many forms, e.g., in the form of a solution, cream, ointment, gel, lotion, shampoo, or aerosol formulation adapted for application to the skin. The weight percent of the active ingredient (e.g., those presented in Table 1, e.g., kaempferol) in the composition useful in preventing or reducing wrinkles or in the treatment of a disorder described herein typically ranges from 0.01% to 10% (e.g., 0.02% 0.05%, 0.1%, 0.2%, 0.5%, 1.0%, 2.0%, 5.0%, or 10%) (based on the total weight of the composition) or from 0.05 μM to 5 mM (e.g., from 0.1 to 10 μM, 0.5 to 50 μM, 1 to 100 μM, 5 to 500 μM, 10 μM to 1 mN, 20 μM to 2 mM, 50 μM to 5 mM, 0.1 to 2 mM, or 0.2 to 1 mM) in admixture with a pharmaceutically acceptable carrier. A wide variety of carrier materials can be employed in the wrinkle reducing composition described herein such as alcohols, aloe vera gel, allantoin, glycerine, vitamin A and E oils, mineral oils, and polyethylene glycols. Other additives, e.g., preservatives, fragrance, sunscreen, or other cosmetic ingredients, can be present in the composition. The topical composition can be applied and removed immediately, or it can be applied and left on the skin surface, e.g., the face, for an extended period of time, e.g., overnight or throughout the day.

### UVB Radiation

The major source of UVB radiation is natural sunlight. The intensity of UVB rays varies depending on the time of day, time of year, the sun's position in the sky, altitude and distance from the equator. These rays are most intense during the midday hours in the summer, although they are always present, even during the winter months. Distance above sea level and distance from the equator are also important to consider. The higher the altitude the greater the intensity of UVB rays. Therefore, mountaineers, skiers, and those who live at high altitudes are at risk of long term UVB damage. Also, the nearer one is to the equator the more intense the UV radiation and the higher the risk of long term UVB damage.

Snow, water, and sand reflect sunlight, magnifying the amount of UVB radiation that reaches the skin. Even when clouds obscure the sun, UVB levels can still be sufficiently high to cause skin damage, e.g., wrinkles, upon long term exposure.

The UV index (developed by the Environmental Protection Agency) indicates the intensity of the sun's UV rays on a given day. There are four categories—moderate (UV index is less than 3), high (UV index is 3 to 6) very high (UV index is 6 to 10) and extreme (UV index is greater than 10). A moderate UV Index means it will take more than an hour to burn skin; an extreme level means it will take less than 15 minutes. The index is often included with weather reports. Clinically, UVB exposure is measured in MEDs. One MED is the amount of UVB required to produce a sunburn in sensitive skin. Because the effects of UVB exposure are cumulative, long term or chronic UVB induced wrinkles can occur as a result of long term exposure to UVB levels below those which, upon acute exposure, can cause erythema or edema or burning (e.g., below one MED). For example, a subject is at risk of long term UVB induced wrinkles if the subject is chronically exposed to the sun even if the subject is only exposed to the sun during days with a low or moderate UV Index.

### Measurement of Wrinkles

The effect of a compound on the formation or appearance of wrinkles can be evaluated qualitatively, e.g., by visual inspection, or quantitatively, e.g., by computer assisted measurements of wrinkle morphology. Preferably, wrinkle morphology is quantitatively analyzed. Examples of quantitative methods for measuring wrinkles include, but are not limited to, the optical cut technique employing a laser beam, as proposed by Hoshino (1992) Pixel 45:121, herein incorporated by a reference; or methods that analyze three-dimensional skin replicas, e.g., the Shiseido Wrinkle Analyzer 3D Pro system (Takasu et al. (1996) J. Soc. Cosmet. Chem. Japan 29:394-405; Japanese Published Patent Application No. 07-113623, published May 2, 1995 (corresponds to U.S. patent application Ser. No. 08/364,346)). The SILFLO® (Flexico Development Ltd., Potters Bar, UK) system or a similar system can be used to take a replica of the skin. Irregularities on the surface of the skin replica, i.e., wrinkles, are analyzed, e.g., with the Shiseido Wrinkle Analyzer 3D Pro or a similar system, to provide three-dimensional shape data from the heights at points on a two-dimensional plane corresponding to the skin. According to the three-dimensional data, the length, width, depth, area, and volume of each wrinkle is calculated. According to the parameters for regular and fine wrinkles described herein, different classes of wrinkles, including the subclasses of regular and fine wrinkles, can thus be individually recognized and scored.

### Kits

An agent that increases RhoE pathway activity, e.g., an agent identified through a method described herein, e.g., a compound presented in Table 1, can be provided in a kit. The kit can include (a) the agent, e.g., a composition that includes the agent, and (b) informational material. The informational material can be descriptive, instructional, marketing, or other material that relates to the methods described herein and/or the use of the agent for the methods described herein. For example, the informational material relates to wrinkles or their prevention or reduction.

In one embodiment, the informational material can include instructions to administer the agent in a suitable manner to perform the methods described herein, e.g., in a suitable dose, dosage form, or mode of administration (e.g., a dose, dosage form, or mode of administration described herein). A preferred dose, dosage form, or mode of administration is topical, e.g., on the skin. In another embodiment, the informational material can include instructions to administer the agent to a suitable subject, e.g., a human, e.g., a human having, or at risk for, wrinkles. For example, the material can include instructions to administer the agent to the face, neck or hands.

The informational material of the kits is not limited in its form. In many cases, the informational material, e.g., instructions, is provide din printed matter, e.g., a printed text, drawing, and/or photograph, e.g., a label or printed sheet. However, the informational material can also be provided in other formats, such as Braille, computer readable material, video recording, or audio recording. In another embodiment, the informational material of the kit is contact information, e.g., a physical address, electronic mail address, web address, or telephone number, where a user of the kit can obtain substantive information about the agent and/or its use in the methods described herein. Of course, the informational material can also be provided in any combination of formats.

In addition to the agent, the composition of the kit can include other ingredients, such as solvent or buffer, a stabilizer, a preservative, a fragrance or other cosmetic ingredient, and/or a second agent for treating a condition or disorder described herein, e.g., a sunscreen. Alternatively, the other ingredients can be included in the kit, but in different compositions or containers than the agent. In such embodiments, the kit can include instructions for admixing the agent and the other ingredients, or for using the agent together with the other ingredients.

The agent can be provided in any form, e.g., liquid, dried, or lyophilized form. It is preferred that the agent be substantially pure and/or sterile. When the agent is provided in a liquid solution, the liquid solution is typically an aqueous solution, e.g., a sterile aqueous solution. When the agent is provided as a dried form, reconstitution generally is by the addition of a suitable solvent. The solvent, e.g., sterile water or buffer, can optionally be provided in the kit.

The kit can include one or more containers for the composition containing the agent. In some embodiments, the kit contains separate containers, dividers, or compartments for the composition and information material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agent. For example, the kit includes a plurality of syringes, ampules, foil packets, or blister packs, each containing a single unit dose of the agent. The containers of the kits can be air tight and/or waterproof.

The kit optionally includes a device suitable for administration of the composition, e.g., a syringe, inhalant, pipette, forceps, measured spoon, dropper (e.g., eye dropper), swab (e.g., a cotton swab or wooden swab), or any such delivery device.

The following specific examples are to be construed as merely illustrative, and not limiting of the remainder of the disclosure in any way whatsoever.

### EXAMPLES

### Example 1

### Identification of RhoE-Inducing Compounds

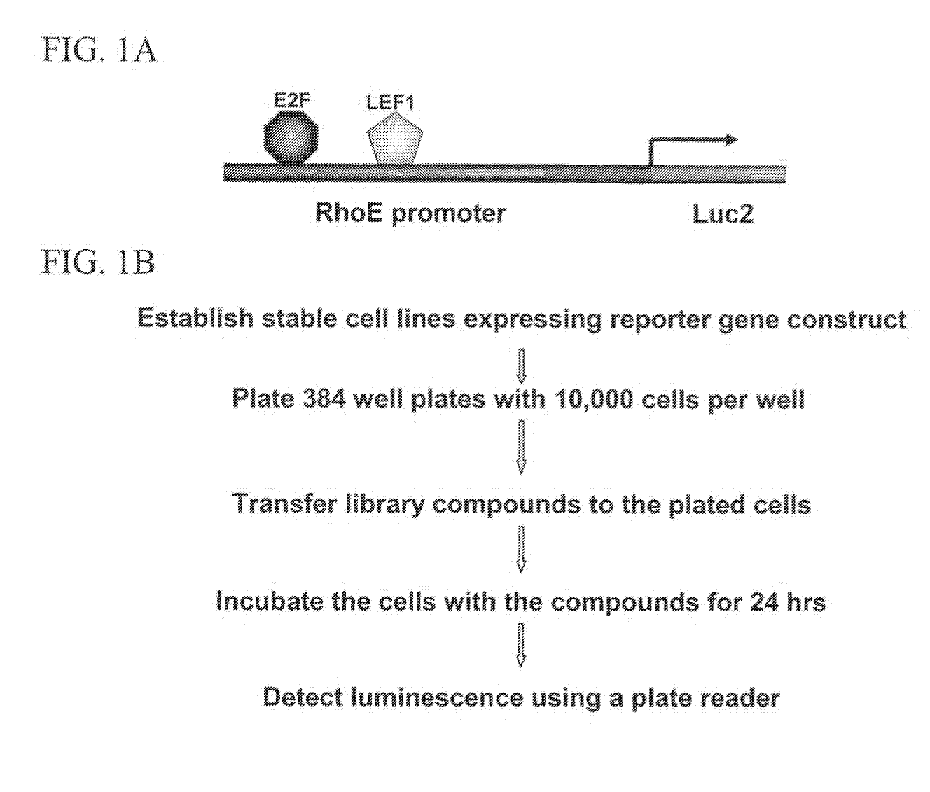

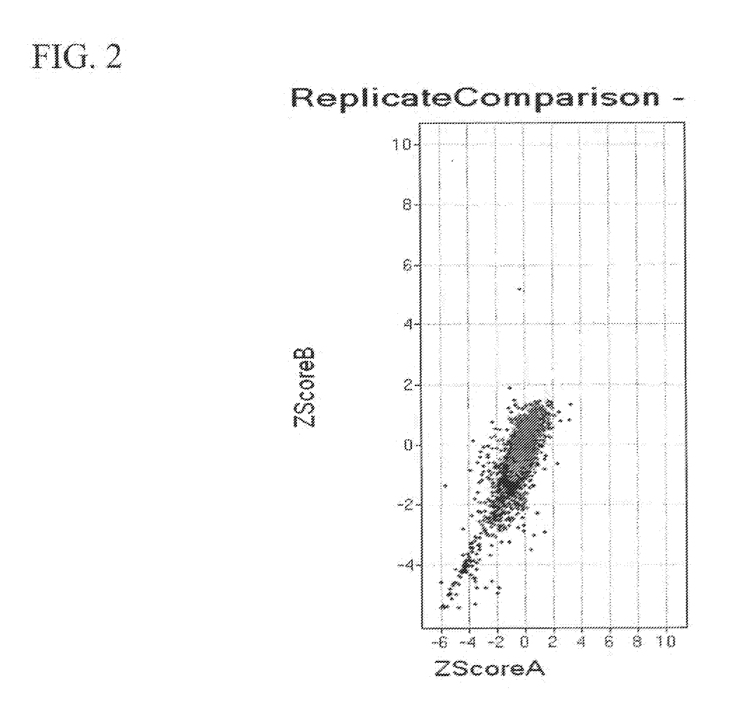

To isolate compounds that can activate RhoE pathway in keratinocytes and protect against UVB exposure, a robust and sensitive chemical genetics approach was developed for the identification of small molecules that induce RhoE expression. An overview of the screening method is presented in FIG. 1B. Briefly, a reporter construct was created that included the RhoE promoter operatively linked to the luc2 luciferase reporter gene (FIG. 1A). Stable cell lines were produced that expressed the reporter construct, and the cells were plated in 384 well plates at ˜10,000 cells per well. The library test compounds were added to the plated cells at two replicates per compound, and the cells were incubated with the compounds for 24 hours. Luminescence was measured at 24 hours using a plate reader. The data were analyzed using Spotfire. Compounds that induced RhoE expression with composite Z values (of two replicates) greater than 2 are presented in Table 1.

TABLE 1
Identified inducers of RhoE expression

| Composite Z value | Compound |
|---|---|
| 6.2959 | Kaempferol |
| 5.8729 | Apigenin |
| 5.4731 | Chrysin |
| 5.4102 | 5,7,4′-trimethoxyflavone |
| 5.3515 | Apigenin |
| 5.1946 | Parthenolide |
| 5.1371 | Parthenolide |
| 4.8646 | Cosmosiin |
| 4.6992 | apigenin triacetate |
| 4.6362 | liquiritigenin dimethyl ether |
| 4.602 | Fenbendazole |
| 4.5672 | Diosmetin |
| 4.5119 | Fenbendazole; fenbendazole |
| 4.5081 | Nabumetone |
| 3.9958 | Lansoprazole |
| 3.977 | acacetin diacetate |
| 3.9036 | Chrysin |
| 3.8701 | Lansoprazole |
| 3.8551 | Nabumetone |
| 3.8448 | Hieracin |
| 3.8383 | 2-methoxyxanthone |
| 3.8036 | 4′-methoxychalcone |
| 3.79 | Xanthone |
| 3.772 | Phenazopyridine hydrochloride |
| 3.6958 | Mebendazole; mebendazole |
| 3.6944 | Mebendazole |
| 3.6564 | biochanin a diacetate |
| 3.651 | Dibenzoylmethane |
| 3.604 | flavokawain b |
| 3.5924 | Tiabendazole |
| 3.4862 | Tilorone |
| 3.477 | chrysin dimethyl ether |
| 3.357 | Rhetsinine |
| 3.2977 | 5,7-dimethoxyisoflavone |
| 3.2901 | biochanin a |
| 3.2375 | Acacetin |
| 3.2205 | suprofen methyl ester |
| 3.1488 | erythromycin stearate |
| 3.1382 | Diphenylurea |
| 3.0721 | 7,4′-dimethoxyisoflavone |
| 3.0534 | Albendazole |
| 3.0257 | 5,2′-dimethoxyflavone |
| 2.9591 | phenazopyridine hydrochloride |
| 2.8734 | n-(9-fluorenylmethoxycarbonyl)-l-leucine |
| 2.8556 | Propachlor |
| 2.8467 | 4′-hydroxychalcone |
| 2.735 | Benzanthrone |
| 2.7326 | 8,2′-dimethoxyflavone |
| 2.7194 | Iridin |
| 2.6831 | Xanthyletin |
| 2.6414 | Ononetin |
| 2.6043 | Butylparaben |
| 2.5938 | Genistein |
| 2.5656 | Chlorpropham |
| 2.473 | Helenine |
| 2.4662 | Euparin |
| 2.4579 | sappanone a 7-methyl ether |
| 2.4062 | 3-methylcholanthrene |
| 2.3862 | dalbergione, 4-methoxy-4′-hydroxy- |
| 2.3814 | Derrustone |
| 2.3759 | benzylhydrazine hydrochloride |
| 2.3583 | hydralazine hydrochloride |
| 2.3429 | Equilin |
| 2.324 | Quinacrine dihydrochloride dihydrate |
| 2.3231 | Naringenin |
| 2.3104 | Ebselen |
| 2.3069 | Luteolin |
| 2.2603 | Tretinoin |
| 2.2516 | Methiazole |
| 2.23 | cuneatin methyl ether |
| 2.2158 | 2-hydroxyanthone |
| 2.2116 | Maackiain |
| 2.21 | 3′,4′-dimethoxyflavone |
| 2.2003 | Oxibendazole |
| 2.164 | 2-ethoxycarbonyl-2-hydroxy-5,7- |
| | dimethoxyiosflavanone |
| 2.1629 | Monobenzone |
| 2.1577 | Rhamnetin |

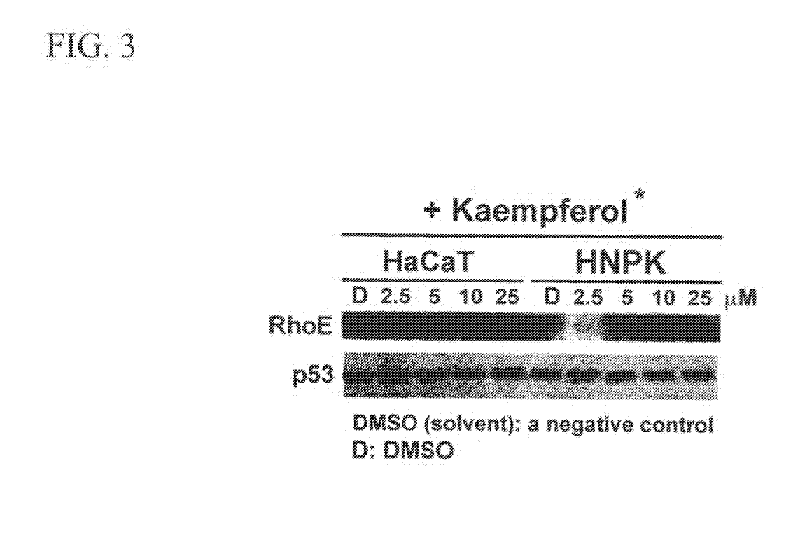

The results were reasonably well reproduced between the two replicates for each compound (FIG. 2). Additional data for the six compounds with the highest composite Z values are presented in Table 2.

TABLE 2
Analysis of identified RhoE inducers

| | Signal | | |
|---|---|---|---|
| Composite Z | to Noise | Reproducibility | Compound |
| 6.2959 | 4.9977 | 0.9781 | Kaempferol |
| 5.8729 | 4.6191 | 0.9872 | apigenin |
| 5.4731 | 4.2497 | 1 | Chrysin |
| 5.4102 | 4.2071 | 0.9985 | 5,7,4′-trimethoxyflavone |
| 5.3515 | 4.1806 | 0.9939 | Apigenin |
| 5.1946 | 4.0526 | 0.9952 | Parthenolide |

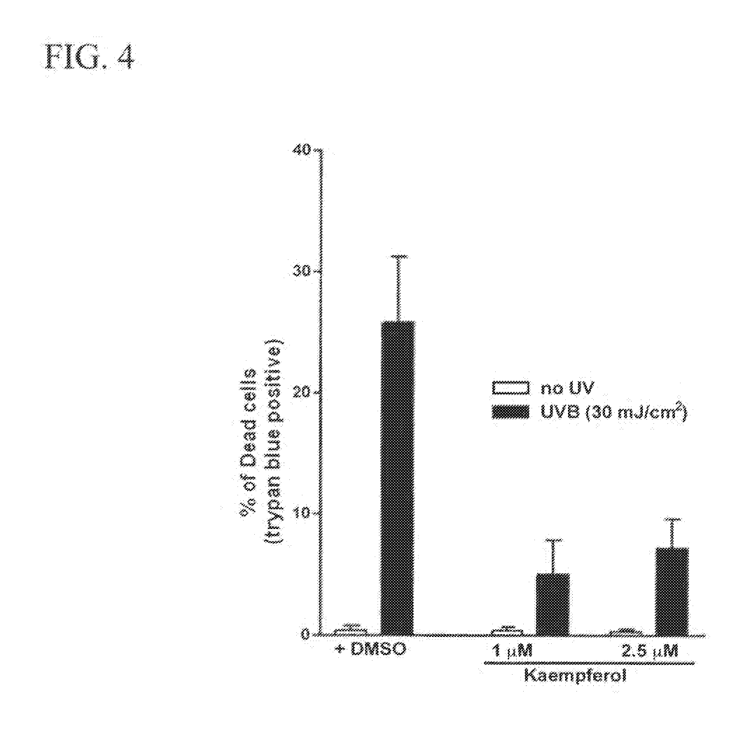

Western blot analysis showed that kaempferol was able to activate RhoE expression in a p53-independent manner, indicating that this compound could be useful as a skin protectant (FIG. 3).

### Example 2

### RhoE-Inducing Compounds Reduce Skin Damage by UVB

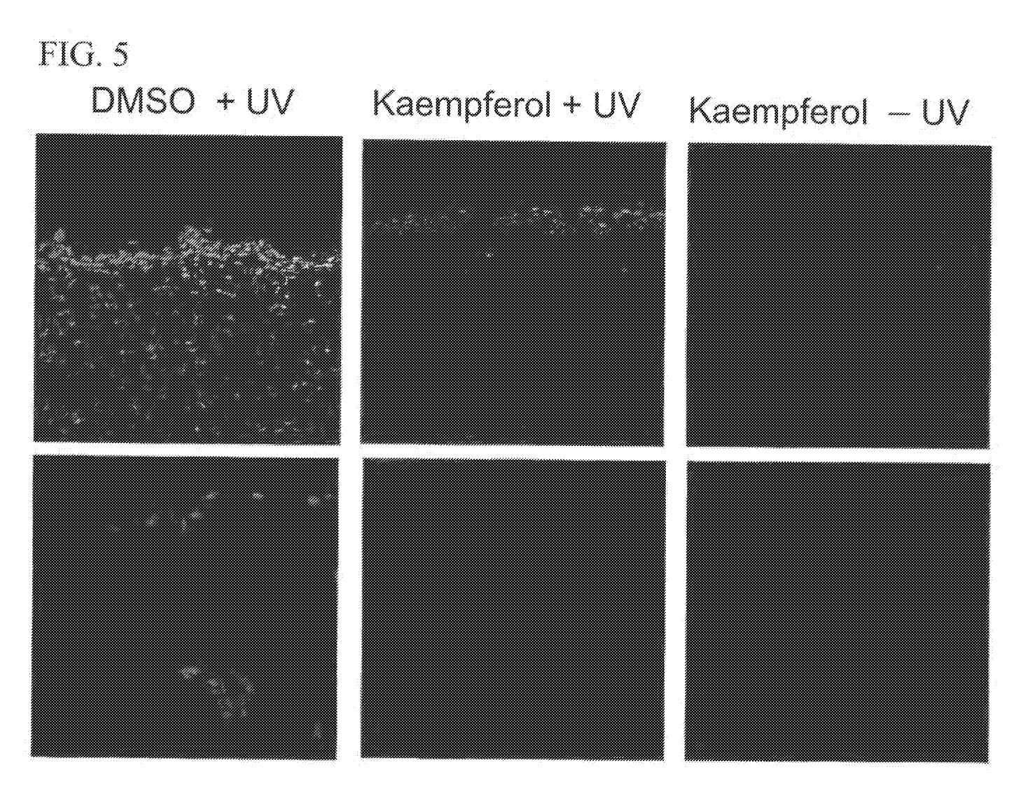

To determine whether RhoE inducing compounds have the protective effect on the UVB damage-mediated cell death, kaempferol-treated (1 and 2.5 μM) HaCaT human keratinocyte cells were exposed to UVB (30 mJ/cm³), and 24 hours later rcell death rate was examined (FIG. 4). Kaempferol treatment resulted in the suppression of UVB-induced cell death in HaCaT cells, suggesting that Kaempferol-mediated RhoE activation protects cells from UVB damage.

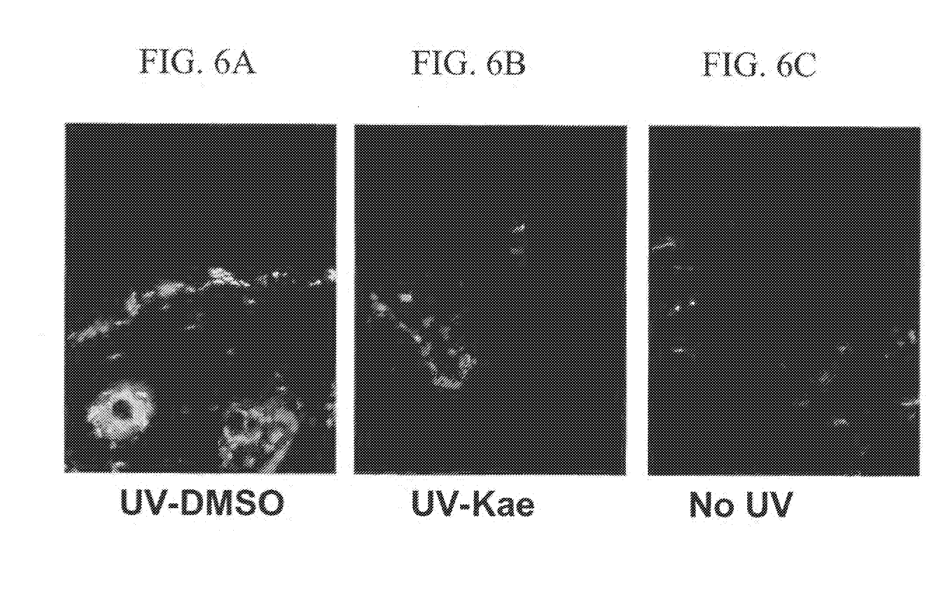

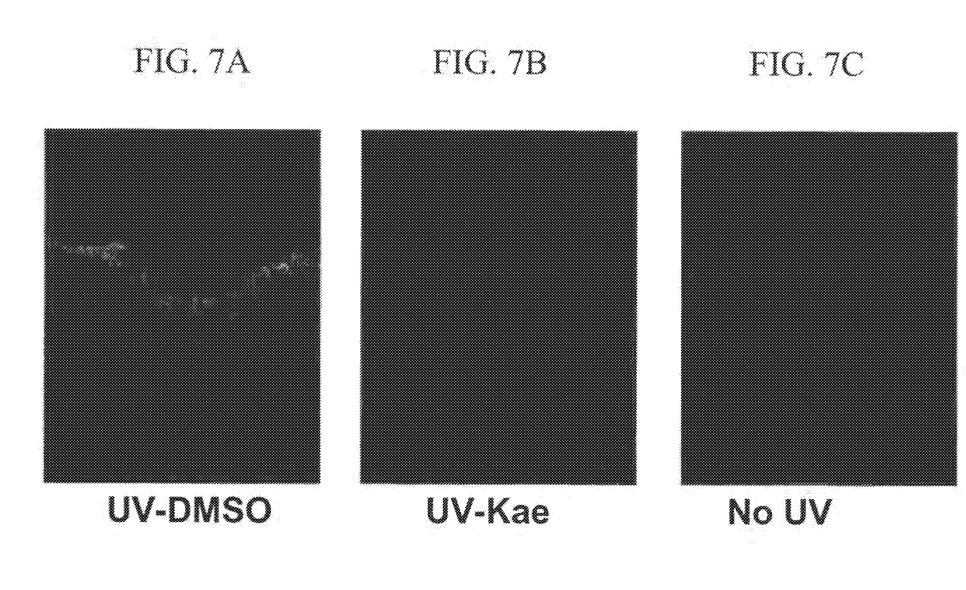

Next, it was investigated whether kaempferol has any protective effect and or DNA repair potential on UVB damaged mouse skin. The dorsal hair of 8-week-old, C57BL/6 mice was trimmed and fine hair was removed by application of a hair-remover, 2 days before UVB exposure. Restrained mice were UVB irradiated at 120 my/cm² and treated with 0.5 mM kaempferol in 50 μl DMSO or DMSO control. The skin was removed from euthanized mice at various times after UVB exposure, fixed immediately in 4% neutral buffered paraformaldehyde, embedded in OCT, and suctioned at 8-μm thickness. Apoptotic cells were detected in skin sections harvested 16 hours after exposure to UVB, by the terminal nucleotidyl transferase-mediated nick end labeling (TUNEL) assay (Roche Applied Science, Germany) according to the manufacture's instructions. Kaempferol-treatment inhibited apoptosis induced by UVB damage in epidermis, as compared to control/DMSO-treated skin (FIG. 5). Moreover, staining with anti-CPD (cyclobutane pyrimidine dimers) monoclonal antibody, clone TDM-2 (MBL International, Woburn, Mass.) (detected using AlexaFlour 488 (Invitrogen, Oregon, USA)) to detect Cyclobutane Pyrimidine Dimers (CPD), a major type of DNA lesion resulting from UV damage showed that Kaempferol treatment suppressed pyrimidine dimer formation generated by UVB exposure in epidermis (FIGS. 6A-6C).

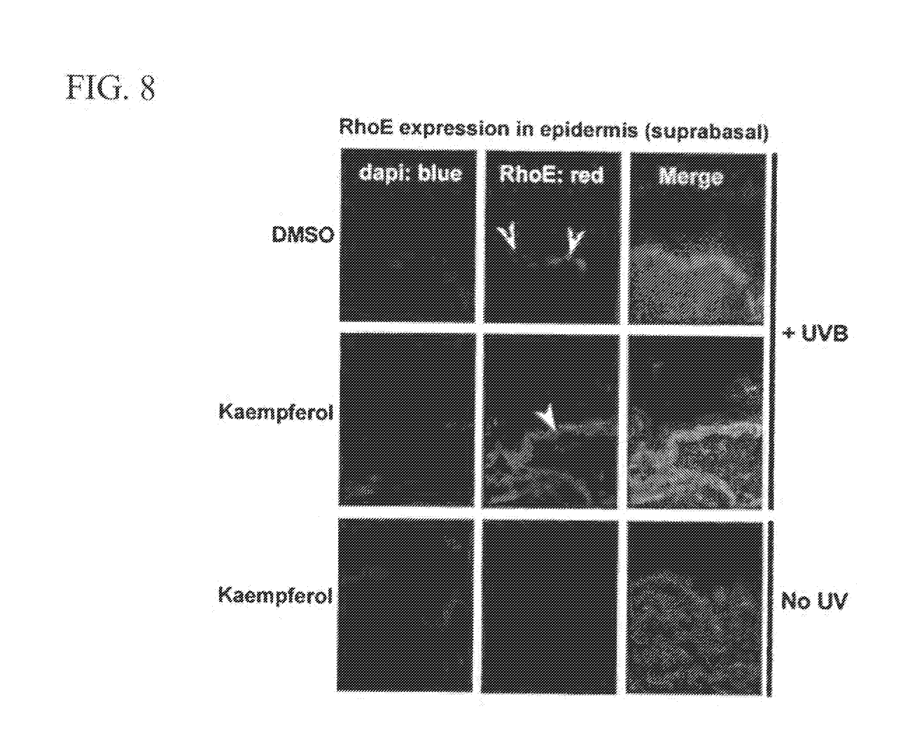

The levels of an activated form of H2AX (Ser139) that is known to be activated and bound at the sites of DNA damage in response to UVB damage were also measured by staining with Anti-Phospho-H2AX (Ser139) antibodies (Upstate Cell Signaling Solutions, New York). The levels of activated form of H2AX (Ser139) in response to UVB damage were significantly decreased in Kaempferol-exposed skin (FIGS. 7A-7C).

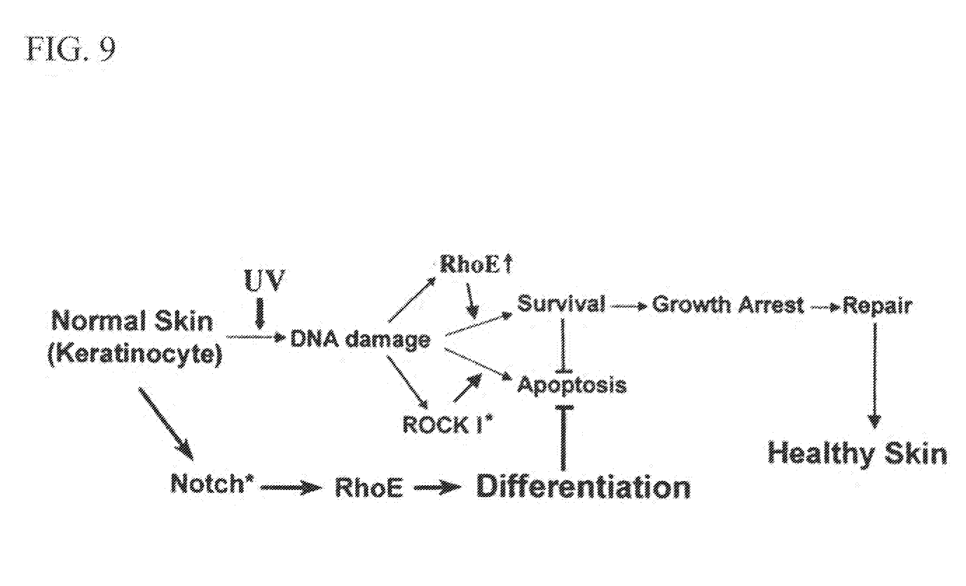

Further, RhoE expression was also enhanced in the suprabasal area of epidermis in Kaempferol-treated skin (FIG. 8). This example indicates that RhoE activation can prevent DNA damage mediated by UVB and enhance DNA repair to maintain healthy skin.

### Example 3

### RhoE is Involved in Notch-Induced Keratinocyte Differentiation

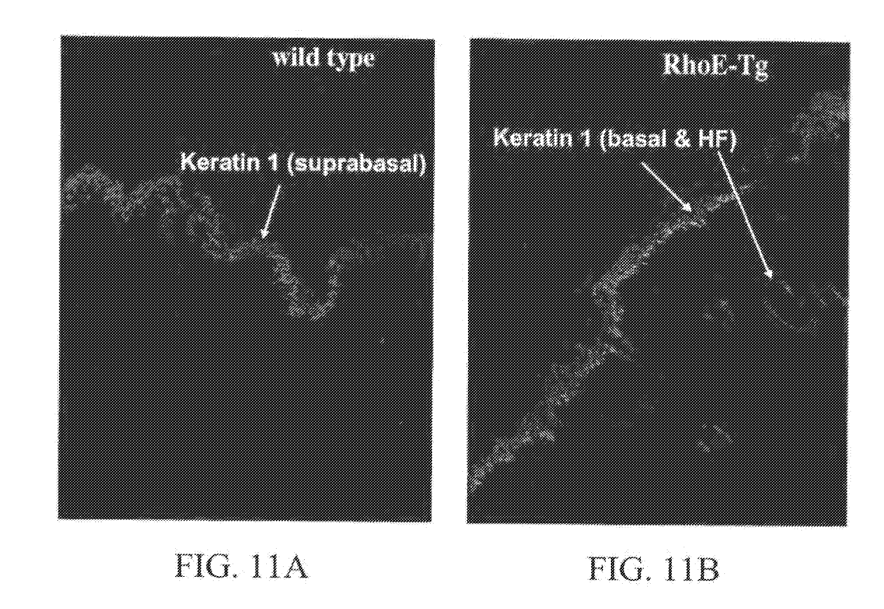

The effects of RhoE on keratinocyte differentiation were determined. RhoE was down- or up-regulated in keratinocytes during Ca²⁺-induced differentiation, and the effects on the differentiation markers cytoplasmic-activated form of Notch1 (NIC) and Involucrin were observed. Down-regulation of RhoE by expression of a shRNA construct (phabe-shRhoE) resulted in decreased expression of NIC, Involuerin, and RhoE as compared to pbabe control vector (FIG. 10A). Conversely, overexpression of RhoE from an adenoviral vector (Ad-RhoE) increased expression of NIC, Involucrin, and RhoE as compared to control adenovirus expressing GFP (Ad-GFP) (FIG. 10B). These results demonstrate that RhoE expression is increased upon Ca²⁺-induced differentiation in mouse keratinocytes.

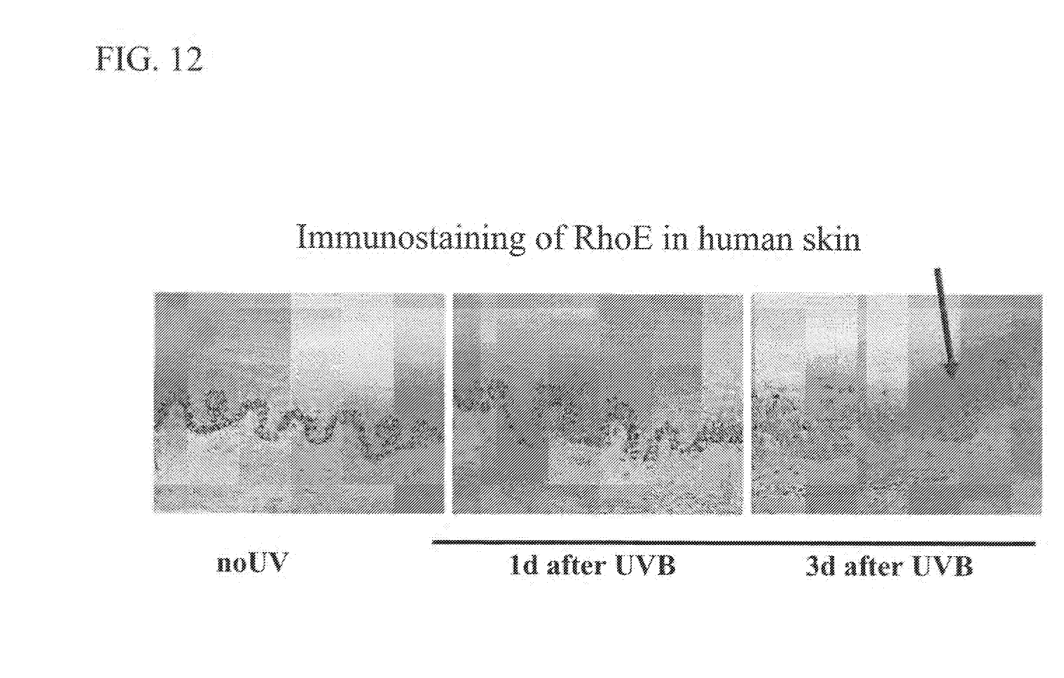

Next the differentiation pattern of the epidermis of transgenic mice expressing the RhoE gene driven by the Keratin 14 promoter was observed. Similar to results seen with Notch1, overexpression of RhoE is able to significantly alter the expression pattern of the differentiation marker Keratin1 (K1). Immunofluorescent staining with K1 specific antibodies showed overexpression of this protein in the basal layer of the epidermis and in the hair follicles, whereas the wild type mice preserve K1 expression only in the suprabasal layer and above in their interfollicular epidermis (FIGS. 11A-B). These findings indicate that RhoE plays a key role in regulating keratinocyte differentiation in vivo and suggest that there is cross-regulation between RhoE and Notch in keratinocyte differentiation.

This example suggests that RhoE is a transcriptional down-stream target of Notch1 and RhoE activation plays a role in Notch-induced keratinocyte differentiation.

### Example 4

### UVB Induces RhoE Expression in Human Skin In Vivo

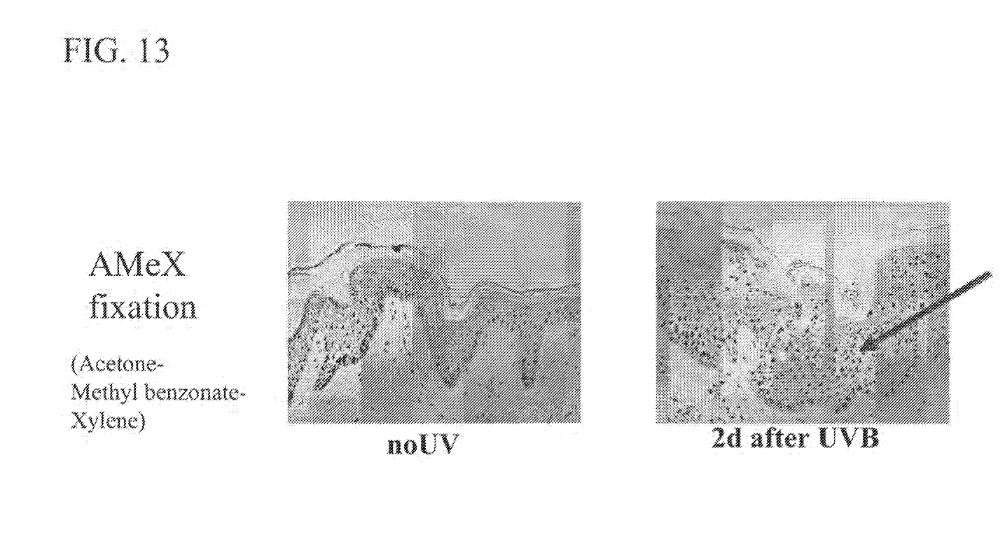

Dorsal skin samples of healthy male volunteers were obtained following UV irradiation and processed by formalin or Acetone-Methyl benzonate-Xylene (AMEX) fixation for detection of RhoE expression. In either formalin (FIG. 12) or AMeX (FIG. 13) fixed tissue, the expression of RhoE was enhanced in superbasal layers of epidermis, but somewhat decreased in basal layers. This suggests that RhoE expression was induced in human keratinocytes by UV DNA damage.

For formalin fixation, the dorsal skin of five healthy male volunteers aged 30-49 years, was UV irradiation with 3 MED (minimal erythema doses) (the MED was determined for each volunteers in advance) using FL-E Lamp (290-320 nm) (Toshiba, Japan) as the UV source. Biopsied skin samples were fixed with 10% formalin and embedded in paraffin. Antigen unmasking was performed by autoclaving at 125° C. for 15 minutes with 1 mM EDTA and 10 mM Tris™ buffer, pH 9. The samples were washed with deionized H₂O three times for 2 minutes. Following washing, the samples were immunostained by incubation for 10 minutes in 0.01% hydrogen peroxide in deionized H₂0 to quench endogenous peroxidase activity. The samples were washed with PBS twice for 5 minutes and then incubated for 30 minutes with 10% normal blocking serum (VECTASTAIN universal elite ABC kit) in PBS. The samples were incubated with primary antibody (anti-RhoE/Rnd3, cloned, Upstate) at 1:200 dilution in PBS with 10% normal blocking serum. After three 5-minute washes with PBS, the samples were incubated with diluted biotinylated secondary antibody solution (VECTASTAIN universal elite ABC kit), washed three more times with PBS for 5 minutes, and incubated for 30 minutes with premixed VECTASTAIN elite ABC reagent. Following three final 5-minute washes with PBS, the cells were incubated with peroxidase substrate (Vector peroxidase substrate kit DAB SK-4100) and rinsed with tap water. Representative samples are shown in FIG. 12.

For AMeX fixation, the dorsal skin of four healthy male volunteers aged 30-40 years was UV irradiated with 2 MED (the MED was determined for each volunteers in advance) using FL-E Lamp (290-320 nm) (Toshiba, Japan) as the UV source. Biopsied skin samples were fixed with cold acetone and embedded in paraffin according to AMeX procedures (Sato et al. (1986) Am. J. Pathol. 125: 431-435). Three micron sections were deparaffinized with xylene and rehydrated. Samples were pretreated with 3% hydrogen peroxide for 15 minutes to block endogenous peroxidase activity. Nonspecific binding was blocked with 10% normal goat serum (Histofine, Tokyo, Japan). The samples were then incubated with primary antibody (anti-RhoE/Rnd3, clone4, Upstate) at a dilution of 1:100 in PBS with 1% bovine serum albumin. After three 5-minute washes with PBS, the samples were incubated with EnVision+system labeled polymer-HRP anti-mouse (DAKO) antibody and washed three more times with PBS. The samples were incubated with Liquid DAB+ substrate-chromogen system (DAKO) and rinsed with tap water. Representative samples are shown in FIG. 13.

### Example 5

### UV Induces Expression of RhoE in Differentiated Keratinocytes In Vitro

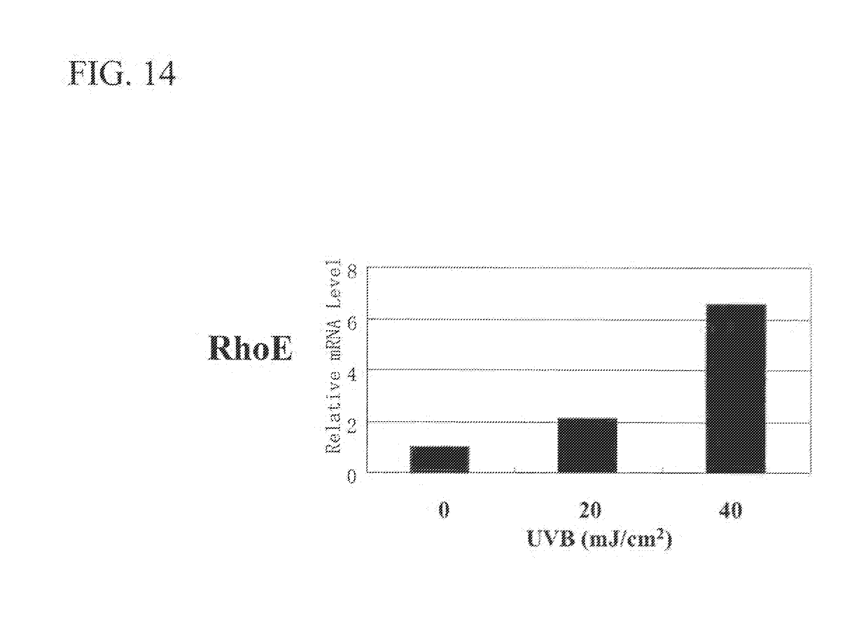

Normal human keratinocytes were cultured until reaching confluency in keratinocyte grown medium (KGM). Differentiation was induced by adding 1.5 mM Ca²⁺, and on the following day the cells were exposed to UVB using FL-E Lamp (290-320 nm) (Toshiba, Japan) as the UV source. 24 hours after UV exposure, cellular RNA was obtained and quantitative RT-PCR was performed using LightCycler to determine RhoE levels. UV irradiation further promoted the gene expression of RhoE in differentiated human keratinocytes (FIG. 14).

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. (canceled)

2. A method of maintaining skin homeostasis in a subject by administering to the subject an agent that increases RhoE pathway activity, thereby maintaining skin homeostasis.

3.-4. (canceled)

5. A method of improving barrier function of the skin of a subject by administering to the subject an agent that increases RhoE pathway activity, thereby improving barrier function of the skin.

6.-7. (canceled)

8. The method of claim 2, wherein the agent increases RhoE activity, induces RhoE expression, decreases Rho kinase I (ROCK I) activity, or reduces ROCK I expression.

9. The method of claim 2, wherein the agent is administered topically.

10. The method of claim 2, wherein the subject has been or will be exposed to UVB radiation.

11. (canceled)

12. The method of claim 2, wherein the agent is selected from Table 1.

13. The method of claim 12, wherein the agent is kaempferol.

14. The method of claim 13, wherein the kaempferol is present at a concentration between 50 μM and 5 mM.

15. A cosmetic composition comprising an agent that increases RhoE pathway activity.

16. The cosmetic composition of claim 15, wherein the composition further comprises a cosmetic ingredient.

17. The cosmetic composition of claim 16, wherein the cosmetic ingredient is a fragrance.

18. The cosmetic composition of claim 16, wherein the cosmetic ingredient is a sunscreen.

19. The cosmetic composition of claim 15, wherein the agent is selected from Table 1.

20. The method of claim 19, wherein the agent is kaempferol.

21. The method of claim 20, wherein the kaempferol is present in the composition at a concentration between 50 μM and 5 mM.

22. The cosmetic composition of claim 15, wherein the composition is formulated for topical application.

23.-33. (canceled)

34. The method of claim 5, wherein the agent increases RhoE activity, induces RhoE expression, decreases Rho kinase I (ROCK I) activity, or reduces ROCK I expression.

35. The method of claim 5, wherein the agent is administered topically.

36. The method of claim 5, wherein the subject has been or will be exposed to UVB radiation.

37. The method of claim 5, wherein the agent is selected from Table 1.
